(19) United States
(12) Patent Application Publication
(10) Pub. No.: US 2024/0350492 A1
(43) Pub. Date: Oct. 24, 2024

(54) Methods for the Prevention and Treatment of Hearing Loss

(71) Applicant: Ting Therapeutics LLC, Omaha, NE (US)

(72) Inventor: Jian Zuo, Omaha, NE (US)

(73) Assignee: Ting Therapeutics LLC, Omaha, NE (US)

(21) Appl. No.: 18/200,148

(22) PCT Filed: Jul. 9, 2021
(86) PCT No.: PCT/US2021/041124

§ 371 Date: May 22, 2023

Related U.S. Application Data
(60) Provisional application No. 63/050,568, filed on Jul. 10, 2020.

(51) Int. Cl. A61K 31/517; A61K 31/167; A61P 27/16

(52) U.S. Cl. CPC A61K 31/517; A61K 31/167; A61P 27/16

(57) ABSTRACT

In one aspect, use of Afatinib as an active agent to treat a hearing loss and to prevent a hearing loss, and methods of treating and/or preventing hearing loss or disorders using the compositions are disclosed. In particular, a method for treating sensorineural hearing loss, including the steps of delivering to a patient in need thereof a composition comprising a therapeutically effective amount of Afatinib. or pharmaceutically acceptable salt thereof is provided. This abstract is intended as a scanning tool for purposes of searching in the particular art and is not intended to be limiting of the present invention.

### CROSS-REFERENCE TO RELATED APPLICATIONS: THIS APPLICATION CLAIMS THE BENEFIT OF U.S

provisional patent application Ser. No. 63/050,568 filed Jul. 10, 2020, under 35 USC § 119 (e) and 35 U.S.C. § 111 (a) (hereby specifically incorporated herein by reference).

Statement regarding federally sponsored research or development: NIH/NIDCD R01DC015444, NIH/NIDCD 1R43 DC018463, Office of Naval Research (ONR) N00014-18-1-2507, Department of Defense (DoD)/USAMRMC-RH170030.

### BACKGROUND OF THE INVENTION

### (1) Field of the Invention

The present invention relates to therapeutic uses of active agent such as for treating, inhibiting, and/or preventing hearing loss.

### (2) Description of Related Art Including Information Disclosed Under 37 CFR 1.97 and 37 CFR 1.98

Over 35 million Americans suffer from hearing loss. In mammals, hair cell loss is permanent. Noise-induced hearing loss (NIHL) affects a quarter of adult humans, many with work obligations in loud environments. The spiral-shaped cochlea of the inner ear is responsible for detecting sound. Inner hair cells lining the cochlea transform the mechanical vibrations of sound waves into chemical signals. These chemicals are then released from the hair cells and received by receptors on the auditory nerve fibers that send electrical impulses to the brain. The junction between a hair cell and a nerve fiber is called a synapse. Loud noise can release too much glutamate, overwhelming the receptors, which leads to loss of synapses and, eventually, a condition called sensorineural hearing loss.

Currently, there are no clinically proven medications for the treatment of hearing loss (sensorineural and neural), or tinnitus associated with the inner ear, and a medication that could be used to prevent, alleviate, or eliminate hearing loss (or tinnitus) would thus be very desirable. The most common remedy for individuals suffering from severe sensorineural hearing loss is a hearing aid, which functions to amplify sound. Hearing aids are non-invasive and can improve an individual's ability to hear. However, hearing aids can often be quite conspicuous and embarrassing to the wearer and hearing aids do not return hearing to normal levels. Furthermore, hearing aids amplify sound indiscriminately, sometimes amplifying sounds that an individual does not wish to hear, such as environmental noise.

There exists a need in the art for a solution to hearing loss due to noise.

### BRIEF SUMMARY OF THE INVENTION

The invention provides a method to prevent or treat hearing loss including the steps of administering to an animal in need thereof an effective amount of a pharmaceutical composition containing a therapeutically active agent, wherein the therapeutically active agent includes: Auranofin, Belinostat, Crizotinib, Dactolisib, Fedratinib, Fedratinib, Lapatinib, Selumetinib, Temsirolimus, Vemurafenib, Vorinostat, AG-1478, NT-113, osimertinib, dacomitinib, AZD3759/zorfertinib, and JCN037/JGK037.

More specifically, the invention provides a method of protecting the inner ear cells from death caused by noise by administering to an animal in need thereof an effective amount of a pharmaceutical composition containing a therapeutically active agent, wherein the therapeutically active agent is selected from the group consisting of: Afatinib, Auranofin, Belinostat, Crizotinib, Dactolisib, Fedratinib, Fedratinib, Lapatinib, Selumetinib, Temsirolimus, Vemurafenib, Vorinostat, AG-1478, NT-113, osimertinib, dacomitinib, AZD3759/zorfertinib, and JCN037/JGK037. Another novel aspect of this invention is a method for treating sensorineural hearing loss, including the steps of delivering to a patient in need thereof a composition made of a pharmaceutically effective amount Afatinib or pharmaceutically acceptable salt thereof.

The novel subject matter includes a composition for use in protecting the inner ear cells from death caused by noise comprising: an effective amount of a pharmaceutical composition containing a therapeutically active agent, wherein the therapeutically active agent is selected from the group consisting of: Afatinib, Auranofin, Belinostat, Crizotinib, Dactolisib, Fedratinib, Fedratinib, Lapatinib, Selumetinib, Temsirolimus, AG-1478, NT-113, osimertinib, dacomitinib, AZD3759, JCN037 Vemurafenib and Vorinostat or a pharmaceutically acceptable salt thereof.

The otoprotective effect of Afatinib was further validated in a mouse model of acute NIHL. Pre-treatment with Afatinib (20 mg/kg/day IP) starting one day before the noise-exposure (octave band noise 8-16 kHz at 100 dB SPL for 2 hours) attenuated permanent threshold shifts (PTS) 14 days post-noise in adult FVB/NJ mice. DPOAE threshold shifts were most prominently protected in Afatinib treated animals at multiple frequencies while ABR threshold shifts were significantly protected at 5.6 and 32 kHz. Cochlear inner hair cell synaptic ribbon counts further supported the protective effect.

### BRIEF DESCRIPTION OF THE SEVERAL VIEWS OF THE DRAWING

FIG. 1 is a schematic diagram showing how compounds of this invention were identified based on a comparison of data sets from drug screen connectivity maps and pathway enrichment analysis reveal drugs acting against hearing loss.

FIG. 2A-D show Afatinib's protective effect against glutamate excitotoxicity in vivo in zebrafish and the absence of additive or synergistic effects of Afatinib in EGF ligand knockdown zebrafish. (A) shows whole cochleae from P28 FVB mice were dissociated and incubated with direct fluorescent antibodies to EpCAM and EGFR. Quarter 2 in the flow plot presents cells that are positive for EGFR. (B) P28 FVB mice were injected transtympanically with DMSO or 20 mg/ml of AG1478. Western blotting was performed on pooled organs of Corti to detect the phosphorylation of EGFR downstream kinases. (C) Zebrafish not injected or injected with 2 ng of scrambled or EGF ligand (EGFL) specific morpholinos (Gene Tools). Animals were pre-incubated with vehicle (PBS) or afatinib (1 μM) for 1 hour followed by 50 min incubation with NMDA (or kainic acid, not shown) (300 μM) to mimic noise exposure. Quantification of the HCs was performed in SO3, O1 and O2 neuromasts. P<0.0001 versus the corresponding control (One-way ANOVA). EGFL morphants did not show any significant differences compared to the corresponding control or the non-injected control. Also, no synergistic or additive effect of afatinib (Afa1) and EGFL knockdown was observed with afatinib application in the morphant. (D) PCR from scrambled and EGFL morphants.

FIG. 3A-3B show adult FVB mice (4-5 weeks old) were exposed to noise trauma (8-16 kHz noise band at 100 dB SPL for 2 hrs) indicated by the shaded box in the figures. (A) ABR threshold shifts 2 weeks after the noise-exposure. Animals treated with 4 doses of Afatinib 20 mg/kg/day IP showed significant difference in the threshold shifts at 16, 22.6, 32 and 45 kHz frequencies when compared to those treated with saline (B) DPOAE thresholds (a function of the outer hair cells of the cochlea). Significant differences in threshold were observed at 11.3, 16 and 22.6 kHz (11.3 & 16 kHz; p<0.0001, 22.6 kHz; p=0.008, 2-way ANOVA with Holm Sidak's multiple comparison). Data are presented as mean±SEM, n=3-4/group. These figures show protection against noise trauma in adult FVB mice when administered Afatinib.

FIG. 4A-B shows compound activity A-B) Vorinostat's protective effect against cisplatin-induced hearing loss A) Levels of caspase-3/7 activity in HEI-OC1 cells treated with Cisplatin (50 μM) and various doses of Vorinostat. Raw caspase reads were normalized to caspase activity in cells treated with Cisplatin/DMSO and cells treated with 1% DMSO. Data are shown as mean±standard error (n=3 wells per treatment). *P<0.05 (One Way ANOVA). B) Levels of protection in zebrafish treated with Cisplatin and experimental compounds quantified by neuromast count hair cell count. Quantification of the HCs at SO3 (supraorbital line neuromast) and O1-2 (Otic line neuromasts) revealed significantly reduced Cisplatin damage in zebrafish HCs pretreated with various doses of Vorinostat (n=5 to 8 per group, One Way ANOVA).

FIG. 5 shows Afatinib reduces noise-induced increase of pAkt and pMAPK in mouse cochleae.

### DETAILED DESCRIPTION OF THE INVENTION

The present invention can be understood more readily by reference to the following detailed description of the invention and the examples included therein. Before the present compounds, compositions, articles, systems, devices, and/or methods are disclosed and described, it is to be understood that they are not limited to specific synthetic methods unless otherwise specified, or to particular reagents unless otherwise specified, as such may, of course, vary. It is also to be understood that the terminology used herein is for the purpose of describing particular aspects only and is not intended to be limiting. Although any methods and materials similar or equivalent to those described herein can be used in the practice or testing of the present invention, example methods and materials are now described.

While aspects of the present invention can be described and claimed in a particular statutory class, such as the system statutory class, this is for convenience only and one of skill in the art will understand that each aspect of the present invention can be described and claimed in any statutory class. Unless otherwise expressly stated, it is in no way intended that any method or aspect set forth herein be construed as requiring that its steps be performed in a specific order. Accordingly, where a method claim does not specifically state in the claims or descriptions that the steps are to be limited to a specific order, it is no way intended that an order be inferred, in any respect. This holds for any possible non-express basis for interpretation, including matters of logic with respect to arrangement of steps or operational flow, plain meaning derived from grammatical organization or punctuation, or the number or type of aspects described in the specification.

In one aspect, an active agent can be used as a therapy for the treatment and/or prevention of hearing loss. In various aspects, the compounds and compositions of the invention can be administered in pharmaceutical compositions, which are formulated according to the intended method of administration. The compounds of this invention are defined as a therapeutically active agent in a treatment regimen or procedure that is intended for preventing hearing loss by noise by protecting inner ear cells from death and in preventing hearing loss from noise. Therapeutic agent means a chemical substance that is used for the treatment or mitigation of a disease condition or ailment.

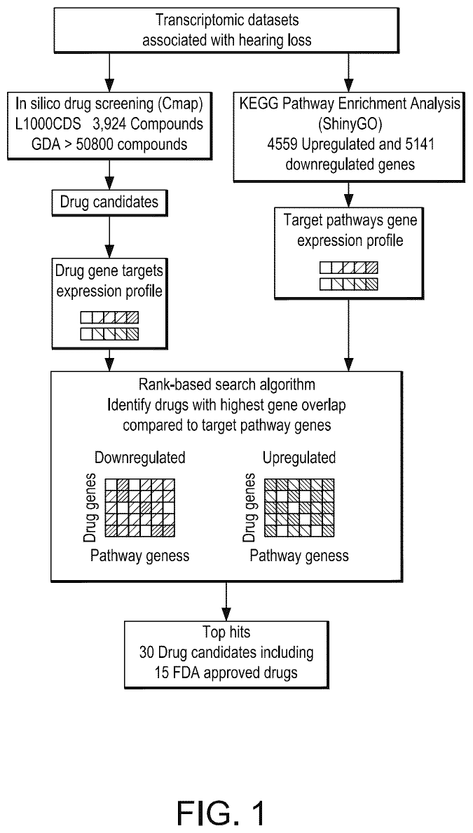

Now referring to FIG. 1 compounds were identified based on a comparison of data sets from drug screen connectivity maps and pathway enrichment analysis reveal drugs acting against hearing loss. This method was developed to derive drug candidates from a diverse chemical space, covering a wide range of biological pathways, avoiding bias associated with focusing on previously reported pathways. First, transcriptomic profiles of unique cell-lines associated with hearing loss are selected which will be used to determine overlaps in gene expression. These cell lines may be sensitive or resistant to hearing loss, have their expression manipulated genetically or by drug-regiments to be associated with hearing loss, or involved in hair cell generation. Second, significantly differentiated pathways from gene expression were identified from the transcriptomic profiles. Separately, also from the transcriptomic profiles, perturbations targeting gene expression and their associated pathway were identified. In this step, because each cell line is unique, significant pathways are those that reveal highly overlapping results to perturbations. Results from both sets were categorized by whether gene expression is up or down regulated. Third, drugs were identified which have high overlap in gene expression with the significantly differentiated pathways to provide a molecular profile of a compound to prevent or treat hearing loss. Here specifically, the data set sought was conformity to a NIHL-resistant mouse stains to NIHL sensitive mouse strains (127SvJ and 86.CAST). The invention provides the compound Afatinib as the active agent, while other compounds with a similar molecular profile can provide protection against noise trauma. These compounds include: Afatinib, Auranofin, Belinostat, Crizotinib, Dactolisib, Fedratinib, Lapatinib, Selumetinib, Temsirolimus, AG-1478, NT-113, osimertinib, dacomitinib, AZD3759, JCN037, Vemurafenib and Vorinostat.

The compounds and compositions described herein can be formulated in a conventional manner using one or more physiologically acceptable carriers or excipients. For example, a pharmaceutical composition can be formulated for local or systemic administration, e.g., administration by drops or injection into the ear, insufflation (such as into the ear), intravenous, topical, or oral administration. Compounds can be synthesized by a variety of methods known in the art.

In one aspect, these compounds can be used as a therapy for the treatment and/or prevention of hearing loss. The invention is directed to non-age-related hearing loss such as tinnitus, sensorineural hearing loss, neural hearing loss, and Meniere's disease. More specifically, sensorineural hearing loss includes noise related hearing loss which can be prevented or treated by administering to an animal in need thereof an effective amount of a pharmaceutical composition containing these compounds.

The term “hearing loss” refers to a defect in the ability to perceive sound and includes partial hearing loss, complete hearing loss, deafness (complete or partial), and tinnitus, the perception of non-existent sounds, i.e., a buzzing in the ear. The hearing loss may be due to hair cell or neuron damage, wherein the damage is caused by a genetic disorder, loud sounds, ototoxicity, or any other such stressor described in the application. Hearing loss includes sensorineural hearing loss, conductive hearing loss, combination hearing loss, mild (between 25 and 40 dB), moderate (between 41 and 55 dB), moderately severe (between 56 and 70 dB), severe (between 71 and 90 dB), and profound (90 dB or greater) hearing loss, congenital hearing loss, pre-lingual and post-lingual hearing loss, unilateral (affecting one ear) and bilateral (affecting both ears) hearing loss, or any combination of these, i.e., sensorineural/severe/postlingual/bilateral.

In various aspects, the compounds and compositions of the invention can be administered in pharmaceutical compositions, which are formulated according to the intended method of administration. The compounds and compositions described herein can be formulated in a conventional manner using one or more physiologically acceptable carriers or excipients. For example, a pharmaceutical composition can be formulated for local or systemic administration, e.g., administration by drops or injection into the ear, insufflation (such as into the ear), intravenous, topical, or oral administration. Afatinib may be synthesized by methods known in the art. Kovacevic, T. et al., An Alternative Synthesis of the Non-Small Cell Lung Carcinoma Drug Afatinib, Tetrahedron Letters Vol. (59), Issue (47). 2018 4180-4182. Reddy, V. Prakash. Organofluorine Compounds in Biology and Medicine. Amsterdam, Netherlands: Elsevier, 2015. 271-272. Compounds may be synthesized by methods known in the art.

Afatinib is revealed to protect against the loss of hair cells. Afatinib is identified as acting against hair cell loss in animals by the models and data presented. Models reveal that Afatinib protects against hair cell loss through interaction with EGFR. Afatinib is revealed to have high efficacy in mouse and zebrafish models used to demonstrate protection against hair cell loss. The lateral-line neuromasts of zebrafish are a valuable model for testing compounds protective against cisplatin toxicity in vivo, as their HCs are considered homologous to those in the mammalian inner ear and are readily accessible to drugs in vivo. Teitz et al., J. Exp. Med. 2; 215 (4): 1187-1203 (2018). Mouse models measuring ABR threshold shift to indicate hearing loss have previously been described. Teitz et al., J. Exp. Med. 2; 215 (4): 1187-1203 (2018). Other compounds such as Auranofin, Belinostat, Crizotinib, Dactolisib, Fedratinib, Fedratinib, Lapatinib, Selumetinib, Temsirolimus, Vemurafenib, Vorinostat, AG-1478, NT-113, osimertinib, dacomitinib, AZD3759/zorfertinib, and JCN037/JGK037 similarly can protect against the loss of hair cells.

The nature of the pharmaceutical compositions for administration is dependent on the mode of administration and can readily be determined by one of ordinary skill in the art. In various aspects, the pharmaceutical composition is sterile or sterilizable. The therapeutic compositions featured in the invention can contain carriers or excipients, many of which are known to skilled artisans. Excipients that can be used include buffers (for example, citrate buffer, phosphate buffer, acetate buffer, and bicarbonate buffer), amino acids, urea, alcohols, ascorbic acid, phospholipids, polypeptides (for example, serum albumin), EDTA, sodium chloride, liposomes, mannitol, sorbitol, water, and glycerol. For example, administration can be parenteral, intravenous, subcutaneous, or oral. Methods for making such formulations are well known and can be found in, for example, Remington's Pharmaceutical Sciences, 18th Ed., Gennaro, ed., Mack Publishing Co., Easton, P A 1990.

In various aspects, the disclosed pharmaceutical compositions include the disclosed compounds (including pharmaceutically acceptable salt(s) thereof) as an active ingredient, a pharmaceutically acceptable carrier, and, optionally, other therapeutic ingredients or adjuvants. The instant compositions include those suitable for oral, rectal, topical, and parenteral (including subcutaneous, intramuscular, and intravenous) administration, although the most suitable route in any given case will depend on the particular host, and nature and severity of the conditions for which the active agent is being administered. The pharmaceutical compositions can be presented in unit dosage form and prepared by any of the methods well known in the art of pharmacy.

In various aspects, the pharmaceutical compositions of this invention can include a pharmaceutically acceptable carrier and a compound or a pharmaceutically acceptable salt of the compounds of the invention. The compounds of the invention, or pharmaceutically acceptable salts thereof, can also be included in pharmaceutical compositions in combination with one or more other therapeutically active compounds. The pharmaceutical carrier employed can be, for example, a solid, liquid, or gas. Examples of solid carriers include lactose, terra alba, sucrose, talc, gelatin, agar, pectin, acacia, magnesium stearate, and stearic acid. Examples of liquid carriers are sugar syrup, peanut oil, olive oil, and water.

In preparing the compositions for oral dosage form, any convenient pharmaceutical media can be employed. For example, water, glycols, oils, alcohols, flavoring agents, preservatives, coloring agents and the like can be used to form oral liquid preparations such as suspensions, elixirs, and solutions; while carriers such as starches, sugars, microcrystalline cellulose, diluents, granulating agents, lubricants, binders, disintegrating agents, and the like can be used to form oral solid preparations such as powders, capsules, and tablets. Because of their ease of administration, tablets and capsules are the preferred oral dosage units whereby solid pharmaceutical carriers are employed. Optionally, tablets can be coated by standard aqueous or nonaqueous techniques. A tablet containing the composition of this invention can be prepared by compression or molding, optionally with one or more accessory ingredients or adjuvants. Compressed tablets can be prepared by compressing, in a suitable machine, the active agent in a free-flowing form such as powder or granules, optionally mixed with a binder, lubricant, inert diluent, surface active or dispersing agent.

The pharmaceutical compositions of the present invention include a compound of the invention (or pharmaceutically acceptable salts thereof) as an active ingredient, a pharmaceutically acceptable carrier, and optionally one or more additional therapeutic agents or adjuvants. The instant compositions include compositions suitable for oral, rectal, topical, and parenteral (including subcutaneous, intramuscular, and intravenous) administration, although the most suitable route in any given case will depend on the particular host, and nature and severity of the conditions for which the active agent is being administered. The pharmaceutical compositions can be conveniently presented in unit dosage form and prepared by any of the methods well known in the art of pharmacy.

Pharmaceutical compositions of the present invention suitable for parenteral administration can be prepared as solutions or suspensions of the active compounds in water. A suitable surfactant can be included such as, for example, hydroxypropylcellulose. Dispersions can also be prepared in glycerol, liquid polyethylene glycols, and mixtures thereof in oils. Further, a preservative can be included to prevent the detrimental growth of microorganisms.

Pharmaceutical compositions of the present invention suitable for injectable use include sterile aqueous solutions or dispersions. Furthermore, the compositions can be in the form of sterile powders for the extemporaneous preparation of such sterile injectable solutions or dispersions. In all cases, the final injectable form must be sterile and must be effectively fluid for easy syringability. The pharmaceutical compositions must be stable under the conditions of manufacture and storage; thus, preferably should be preserved against the contaminating action of microorganisms such as bacteria and fungi. The carrier can be a solvent or dispersion medium containing, for example, water, ethanol, polyol (e.g., glycerol, propylene glycol and liquid polyethylene glycol), vegetable oils, and suitable mixtures thereof.

In addition to the aforementioned carrier ingredients, the pharmaceutical formulations described above can include, as appropriate, one or more additional carrier ingredients such as diluents, buffers, flavoring agents, binders, surface-active agents, thickeners, lubricants, preservatives (including antioxidants) and the like. Furthermore, other adjuvants can be included to render the formulation isotonic with the blood of the intended recipient. Compositions containing a compound of the invention, and/or pharmaceutically acceptable salts thereof, can also be prepared in powder or liquid concentrate form.

A pharmaceutically effective amount is the dosage for both prophylaxis and treatment without undesirable side effects, such as toxicity, irritation, or allergic response. Although individual needs may vary, the determination of optimal ranges for effective amounts of formulations is within the skill of the art. Human doses can readily be extrapolated from animal studies (Katocs et al., (1990) Chapter 27 in Remington's Pharmaceutical Sciences, 18th Ed., Gennaro, ed., Mack Publishing Co., Easton, PA). In general, the dosage required to provide an effective amount of a formulation, which can be adjusted by one skilled in the art, will vary depending on several factors, including the age, health, physical condition, weight, type and extent of the disease or disorder of the recipient, frequency of treatment, the nature of concurrent therapy, if required, and the nature and scope of the desired effect(s) (Nies et al., (1996) Chapter 3, In: Goodman & Gilman's The Pharmacological Basis of Therapeutics, 9th Ed., Hardman et al., eds., McGraw-Hill, New York, NY).

The following are intended to be exemplary of how one of ordinary skill in the art could make and evaluate the claimed methods compounds, compositions, articles, and/or devices, and are not intended to limit the scope of the invention.

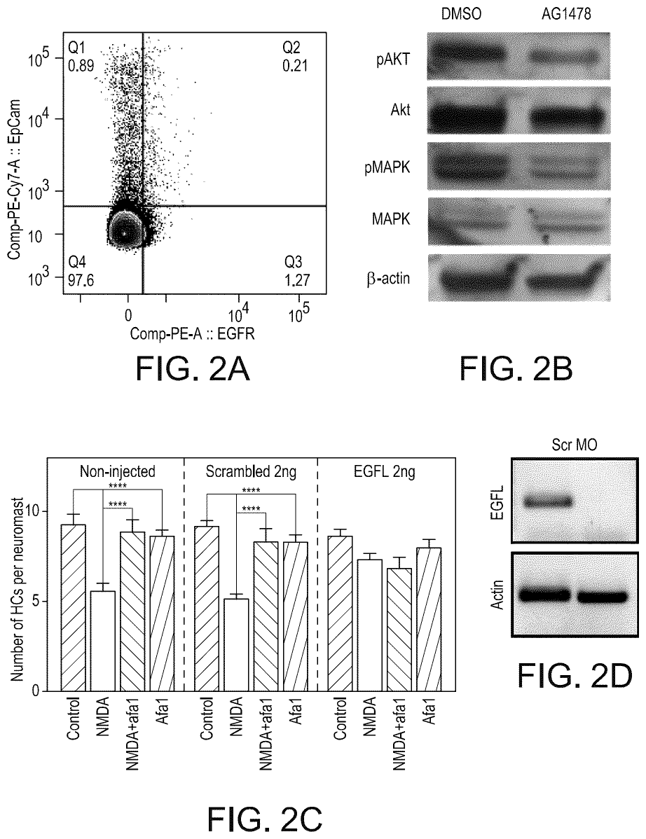

Now referring to FIGS. 2A-D, to determine whether functional EGFR is expressed in the adult cochlea, flow cytometry was used to identify epithelial cells that express EGFR protein in the adult cochlea (FIG. 2A). In P28 FVB mice, excluding dead cells and those with autofluorescence, we detected 1140±88 EGFR+; EpCAM+ (an epithelial cell marker) cells from one cochlea (Mean±SEM, n=4). Furthermore, AG1478, a 1st generation of EGFR inhibitor, was delivered into the adult mouse cochleae through middle ear trans tympanic injection, EGFR downstream signaling pathways (AKT and MAPK) were significantly suppressed in the organ of Corti in vivo (FIG. 2B). These results strongly indicate that EGFR is expressed in the adult organ of Corti, and the receptors are functional and responsive to the inhibitor.

Afatinib is an FDA-approved dual epidermal growth factor receptor (EGFR) and human epidermal growth factor receptors 2/4 (HER2/4) inhibitor. It is FDA-approved as the first-line treatment for patients with advanced/metastatic non-small cell lung cancer carrying EGFR mutations. Afatinib inhibits the non-mutant forms of EGFR and HER2 with IC50 of 0.5 and 14 nM, respectively, with excellent kinase selectivity.

Afatinib was tested for protection against glutamate excitotoxicity in vivo in zebrafish under an approved animal protocol by Creighton University. To measure Afatinib's protection against excitotoxic damage in vivo, zebrafish was exposed to NMDA 300 uM for 50 min followed by a 2 hour incubation with Afatinib. At the end of the experiments, animals were transferred to fish water, fixed and immunostained for the hair cell marker, otoferlin. Hair cells were counted under a fluorescence microscope. Zebrafish may be pretreated with 1.0 μM Afatinib. The fish may be incubated with or without test compounds. NMDA was used to evoke glutamate excitotoxicity The zebrafish may be examined at 5 days after fertilization. The fish may be imaged by epifluorescence microscope. Neuromasts in each fish may be scored based on their staining intensity or the number of cells per neuromast may be counted. NMDA was administered in a concentration of 300 μM to evoke glutamate excitotoxicity. When the zebrafish lateral line was post-incubated with 1 μM of Afatinib, neuromast hair cells were protected from glutamate excitotoxicity. The procedure was run on EGF ligand knockdown zebrafish. A reduction in glutamate excitotoxicity was found. Notably, there was an absence of additive or synergistic effects of Afatinib in EGF ligand knockdown zebrafish. Because there is no established cochlear explant assay that mimics noise injury, we adopted a zebrafish lateral line assay using glutamate excitotoxicity (74). Previous studies have shown that NIHL may be caused, in part, by glutamate excitotoxicity (75-77). Ionotropic glutamate receptor agonists have been used to mimic noise exposure in zebrafish larvae (74). We therefore tested the efficacy of Afatinib to protect against glutamate excitotoxicity in this zebrafish lateral line neuromast model. Pre-incubation with afatinib (1M) protected the neuromast hair cells from damage by 300 μM N-methyl-D-aspartate (NMDA) evoked glutamate excitotoxicity (FIG. 2C). To provide evidence that afatinib acts through the EGFR signaling pathway, we tested morpholino knockdown (KD) of the EGF ligand in zebrafish (FIG. 2D) and a similar protection was observed with 1 μM afatinib (FIG. 2C). Interestingly, when afatinib and EGF ligand knockdown were combined, no synergistic or additive effect was noticed, suggesting that the protective effect is mediated via EGFR pathway and that EGFR is the major pathway by which afatinib protects against glutamate excitotoxicity.

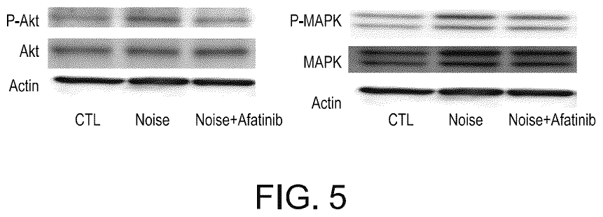

Similarly, now referring to FIG. 5, FVB adult mice were treated with afatinib (IP) two days and noise exposed one day after the 1st afatinib. Cochlear lysates were collected 30 min after noise exposure. The data shows that Afatinib reduces noise-induced increase of pAkt and pMAPK in mouse cochleae.

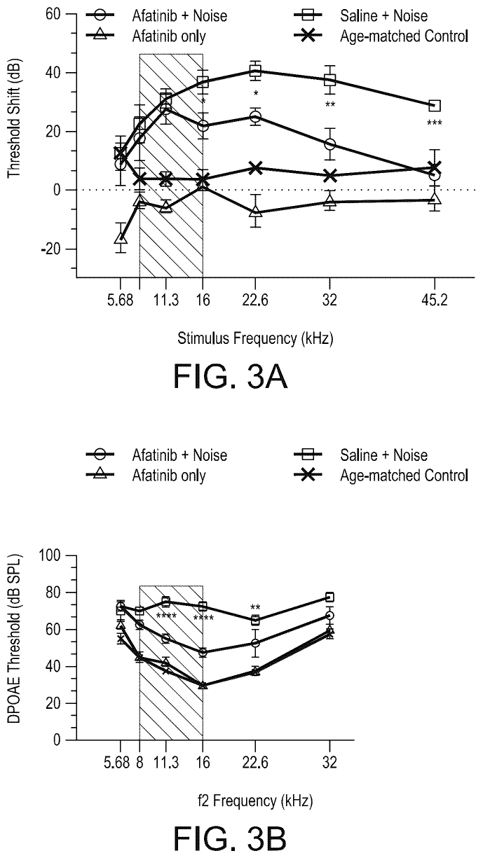

Now referring to FIGS. 3A and 3B, to confirm that Afatinib protects against noise-induced hearing loss in vivo a set of experiments were performed. Adult FVB mice were tested for Afatinib's protection against hearing loss by measuring ABR threshold shifts in the mouse ear. Wild-type FVB mice will be used at age P28, when hearing has matured but long before significant age-related hearing loss. Kermany M H. et al., Hear Res. 2006 October; 220 (1-2): 76-86.; Maison S F. et al., J. Neurosci. 2002 Dec. 15; 22 (24): 10838-46; Maison S F. et al., J. Neurophysiol. 2007 April; 97 (4): 2930-6; Zheng Q Y. et al., Hear Res. 1999 April; 130 (1-2): 94-107. The standard noise exposure protocols (94, 100-, 106-, 116-, and 120-dB sound pressure level (SPL) octave-band 8-16 kHz noise for 2 hrs) have previously been tested in various transgenic mouse strains from the FVB background. Maison S F. et al., J. Neurosci. 2002 Dec. 15; 22 (24): 10838-46; Maison S F. et al., J. Neurophysiol. 2007 April; 97 (4): 2930-6.

Animals were anesthetized with 2,2,2-tribromoethanol (375 μg/g bw, ip) and were exposed to an 8-16 kHz octave band noise at 100 db SPL for 2 hrs. Subcutaneous needle electrodes were inserted behind the pinna (inverting), vertex of the skull (non-inverting) and base of the tail (ground). Tone bursts of 5 ms duration with 0.5 ms cosine-squared envelopes delivered at a rate of 21 stimuli per second with alternating polarity were generated using BioSigRZ software and RZ6 multi I/O processor system (Tucker-Davis Technologies, FL). Stimuli were presented as open field via a speaker (MF1; TDT, FL) placed 10 cm in front of the pinna of the animal. Evoked responses were amplified (20×), bandpass filtered (300-3,000 Hz) and average of 512 responses of 10 ms duration was recorded. Stimulus intensity was decreased in 5 dB increments, starting from 100 db SPL to 0 dB SPL. Thresholds at 5.6, 8, 11.3, 16, 22.6, 32, and 45.2 kHz were identified by visual inspection from stacked waveforms as the lowest level at which reproducible response could be identified. Before the start of every session, stimulus presenting speaker (MF1) was calibrated with a ¼″ microphone (PCB-378C10; PCB Piezotronics, NY) that was also placed 10 cm in front of the speaker. Similar experiments have been previously described in Rai V. et al., Sci Rep. 2020 Sep. 16; 10 (1): 15167.

Adult FVB mice were administered 4 doses of Afatinib 20 mg/kg/day IP. Mice were exposed to noise trauma by 8-16 kHz noise band at 100 dB SPL for 2 hrs. Auditory brainstem response (ABR) thresholds were recorded at P28 in adult FVB wild-type mice and exposed to 100 dB SPL 8-16 kHz octave band noise for 2 hrs. Afatinib was injected in four doses IP at concentration 20 mg/kg/day. Another group of mice were administered saline. Significant differences in threshold were observed at 11.3, 16 and 22.6 kHz (11.3 & 16 kHz; p<0.0001, 22.6 kHz; p=0.008, 2-way ANOVA with Holm Sidak's multiple comparison). Data are presented as mean±SEM, n=3-4/group. ABR and DPOAE thresholds were recorded prior, 7 days, or 14 days post noise exposure.

Adult FVB mice were tested for Afatinib's protection against hearing loss by measuring DPOAE threshold in the mouse ear. After noise trauma and administration of Afatinib, a reduction in DPOAE thresholds was revealed. Adult FVB mice (4-5 weeks old) were exposed to noise trauma (8-16 kHz noise band at 100 dB SPL for 2 hrs) indicated by the shaded box in the figures. Animals treated with 4 doses of Afatinib 20 mg/kg/day IP showed significant difference in DPOAE threshold shifts at 11.3 kHz & 16 kHz (p<0.0001), and 22.6 kHz (p=0.008) frequencies when compared to those treated with saline.

The in vivo set of experiments were conducted to test two additional groups of mice. One group of mice were not exposed to noise or administered Afatinib; this set of mice were used as an age-matched control. Another set of mice were given the same dose of Afatinib in the absence of noise. It was found that threshold shift was significantly decreased when mice were unexposed to noise trauma and given Afatinib compared to the age-matched control, the mice exposed to noise and Afatinib, and the saline and noise trauma exposed mice control group. These groups of mice were additionally tested for DPOAE threshold. Both the group exposed to no noise trauma and given Afatinib and the age-matched control group showed reduced DPOAE threshold at all f2 frequencies compared to the saline and noise trauma control group and the Afatinib and noise trauma group. DPOAE thresholds (a function of the outer hair cells of the cochlea). Significant differences in threshold were observed at 11.3, 16 and 22.6 kHz (11.3 & 16 kHz; p<0.0001, 22.6 kHz; p=0.008, 2-way ANOVA with Holm Sidak's multiple comparison). Data are presented as mean #SEM, n=3-4/group.

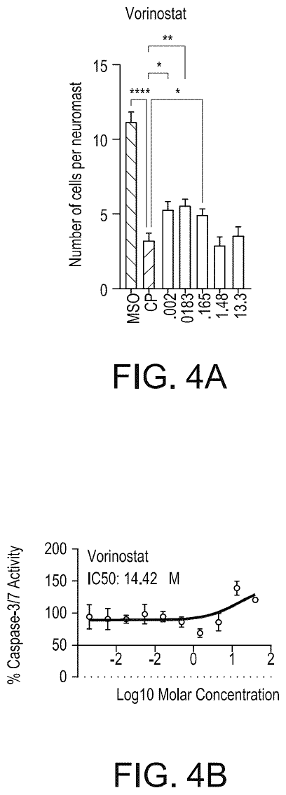

Now referring to FIG. 4A-B, shows Vorinostat demonstrates otoprotective effects against cisplatin. Now referring to FIG. 4A, Vorinostat protects against CIHL. Caspase activity was measured using Caspase-Glo 3/7 assay and then the results were calculated as percentage of protection as an indicator of cell survival/viability. Percent protection for each compound at the tested dosages were then plotted to show dose response curves, and IC₅₀s were calculated. Vorinostat had a relatively low calculated IC₅₀ of 14.42 μM. FIG. 4A.

Now referring to FIG. 4B, Vorinostat protects against cisplatin ototoxicity across multiple doses in zebrafish (n=5-8 per group, One Way ANOVA). *P<0.05, data are shown as mean±standard error (n=5 per group). *P<0.05, data shown as mean±standard error in all panel. Zebrafish were incubated with Vorinostat at 0.002, 0.018, 0.165, 1.48, and 13.3 UM for 1 hour followed by co-incubation with 400 UM cisplatin for 4 hours. At doses of 0.002, 0.0183, and 0.165 μM Vorinostat showed protection against CIHL.

All publications, patents and patent applications mentioned in this specification are herein incorporated by reference to the same extent as if each individual publication, patent, or patent application was specifically and individually indicated to be incorporated by reference.
Teitz T, Fang J, Goktug A N, Bonga J D, Diao S, Hazlitt R A, Iconaru L, Morfouace M, Currier D, Zhou Y, Umans R A, Taylor M R, Cheng C, Min J, Freeman B, Peng J, Roussel M F, Kriwacki R, Guy R K, Chen T, Zuo J. CDK2 Inhibitors as Candidate Therapeutics for Cisplatin- and Noise-Induced Hearing loss. J Exp Med. 2018 Apr. 2; 215 (4): 1187-1203.
Zou J, Yang X, Wei X. Restricted localization of ponli, a novel zebrafish MAGUK-family protein, to the inner segment interface areas between green, red, and blue cones. Invest Ophthalmol Vis Sci. 2010 March; 51 (3): 1738-46.
Kermany M H, Parker L L, Guo Y K, Miller D, Swanson D J, Yoo T J, Goldowitz D, Zuo J. Identification of 17 hearing impaired mouse strains in the TMGC ENU-mutagenesis screen. Hear Res. 2006 October; 220 (1-2): 76-86.
Maison S F, Luebke A E, Liberman M C, Zuo J. Efferent protection from acoustic injury is mediated via alpha9 nicotinic acetylcholine receptors on outer hair cells. J Neurosci. 2002 Dec. 15; 22 (24): 10838-46.
Maison S F, Parker L L, Young L, Adelman J P, Zuo J, Liberman M C. Overexpression of SK2 channels enhances efferent suppression of cochlear responses without enhancing noise resistance. J Neurophysiol. 2007 April; 97 (4): 2930-6.
Zheng Q Y, Johnson K R, Erway L C. Assessment of hearing in 80 inbred strains of mice by ABR threshold analyses. Hear Res. 1999 April; 130 (1-2): 94-107.
Rai V, Wood M B, Feng H, Schabla N M, Tu S, Zuo J. The immune response after noise damage in the cochlea is characterized by a heterogeneous mix of adaptive and innate immune cells. Sci Rep. 2020 Sep. 16; 10 (1): 15167.
Kovacevic T, Mesic M, Avdagic A, Zegarac M. An alternative synthesis of the non-small cell lung carcinoma drug Afatinib, Tetrahedron Letters Vol. (59), Issue (47). 2018 4180-4182.
Reddy, V. Prakash. Organofluorine Compounds in Biology and Medicine. Amsterdam, Netherlands: Elsevier, 2015. 271-272.

While the invention has been described with reference to details of the illustrated embodiments, these details are not intended to limit the scope of the invention as defined in the appended claims. The embodiment of the invention in which exclusive property or privilege is claimed is defined as follows:

What is claimed:

1. A method of protecting ear cells from death caused by noise exposure comprising: administering to a mammal in need thereof an effective amount of a pharmaceutical composition containing a therapeutically active agent, wherein the therapeutically active agent is Afatinib.

2. (canceled)

3. A method to prevent hearing loss from an ototoxic event comprising: administering to a mammal in need thereof an effective amount of a pharmaceutical composition containing a therapeutically active agent, wherein the therapeutically active agent is Afatinib, wherein the ototoxic event is caused by a chemotherapeutic agent.

4. (canceled)

5. (canceled)

6. (canceled)

7. (canceled)

8. A kit comprising: an active agent, wherein the active agent is Afatinib, or a pharmaceutically acceptable salt thereof; and one or more of:
(A) an at least one chemotherapeutic agent;
(B) an at least one antibiotic; and
(C) instructions for preventing a hearing impairment.

9. (canceled)

10. The kit of claim 8, wherein the at least one chemotherapeutic agent is cisplatin.
